(19) United States
(12) Patent Application Publication
(10) Pub. No.: US 2024/0009003 A1
(43) Pub. Date: Jan. 11, 2024

(54) INSTRUMENT HOLDER ASSEMBLIES AND RETRACTORS FOR SPINAL SURGERY PROCEDURES

(71) Applicant: Amplify Surgical, Inc., Irvine, CA (US)

(72) Inventors: Pako Barba, San Diego, CA (US); Andy Choi, Irvine, CA (US); Ali Araghi, Scottsdale, AZ (US)

(21) Appl. No.: 18/470,140

(22) Filed: Sep. 19, 2023

Related U.S. Application Data
(63) Continuation of application No. PCT/US2022/021193, filed on Mar. 21, 2022.
(60) Provisional application No. 63/163,489, filed on Mar. 19, 2021.

(51) Int. Cl. A61F 2/46; A61B 17/02; A61F 2/44; A61B 17/34

(52) U.S. Cl. CPC A61F 2/4611; A61B 17/02; A61F 2/4455; A61B 17/3423; A61F 2002/4615; A61B 2017/3454; A61F 2002/4687

(57) ABSTRACT

In some embodiments, a spine surgical system includes an access tool defining a lumen configured to receive one or more tools or instruments. The spine surgical system can further include one or more tool guides insertable within the access tool lumen and configured to wrap around and keep at least one of the tools during insertion and prevent, inhibit, or limit translation during implantation, device expansion, etc. Each of the tool guides can be designed/customized based at least partially on, or otherwise configured to correspond to, at least one of the instruments. Additionally, or alternatively, the access tool body can include an expandable configured to flex or bend in response to the insertion of one or more tools, such as insertable expanders, within the access tool lumen.

### CROSS-REFERENCE TO RELATED APPLICATIONS

This application is a continuation of International Application No. PCT/US2022/021193, filed on Mar. 21, 2022, titled “INSTRUMENT HOLDER ASSEMBLIES AND RETRACTORS FOR SPINAL SURGERY PROCEDURES,” which claims the benefit of U.S. Provisional Patent Application No. 63/163,489, filed on Mar. 19, 2021, titled “RETRACTOR,” and to U.S. Provisional Patent Application No. 63/163,521, filed on Mar. 19, 2021, titled “EXPANDABLE AND TAPERED RETRACTORS.” Each of these applications is hereby incorporated by reference herein in its entirety.

### TECHNICAL FIELD

The present technology relates generally to the field of surgery, and more particularly, to retractors for spinal surgery procedures, and associated devices, systems, and methods.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a side view of a spinal surgical system positioned along a human subject's spine and configured in accordance with embodiments of the present technology.

FIGS. 2A and 2B are side views of an intervertebral spacer positioned in an intervertebral space and an instrument holder assembly configured in accordance with embodiments of the present technology.

FIGS. 3-5B are isometric views of a spinal surgical system including an instrument holder assembly configured in accordance with embodiments of the present technology.

FIGS. 6A and 6B are perspective views of a tapered access cannula configured in accordance with embodiments of the present technology.

FIGS. 7A-7D are perspective views of an expandable retractor assembly configured in accordance with embodiments of the present technology.

FIGS. 8A-8C are perspective views of an adjustable retractor assembly configured in accordance with embodiments of the present technology.

FIG. 9 is a top view of tools for use in a spinal surgical procedure in accordance with embodiments of the present technology.

FIG. 10 is a schematic top plan view showing surgical approaches to a lumbar spine for performing interbody fusion procedures.

FIG. 11 is an isometric view of the lumbar spine of FIG. 10.

### DETAILED DESCRIPTION

In some embodiments, a spine surgical system includes instrument holder assemblies defining lumens configured to receive one or more tools or instruments. The instrument holder assemblies can include one or more tool guides insertable within an access tool lumen and configured to wrap around and keep an inserter positioned during insertion and prevent, inhibit, or limit translation during implantation, device expansion, etc. For example, the tool guide can inhibit or prevent relative movement (e.g., slipping) during initial docking. This allows a user to find anatomical features (e.g., spinal features, disc, interspinous spaces, intervertebral spaces, etc.) using, for example, fluoroscopy or other visualization techniques. Each of the tool guides can be designed/customized based at least partially on, or otherwise configured to correspond to, at least one of the instruments. The access tools can include guidewires, dilators, cannulas, retractors, clamps, and other tools for accessing surgical sites along the spine. Additionally, or alternatively, an access tool can be a retractor with an expandable portion configured to flex or bend in response to the insertion of one or more tools, such as insertable expanders, within the access tool lumen. In these and other embodiments, the access tool can be a retractor that is tapered (e.g., radially inward) or conically shaped.

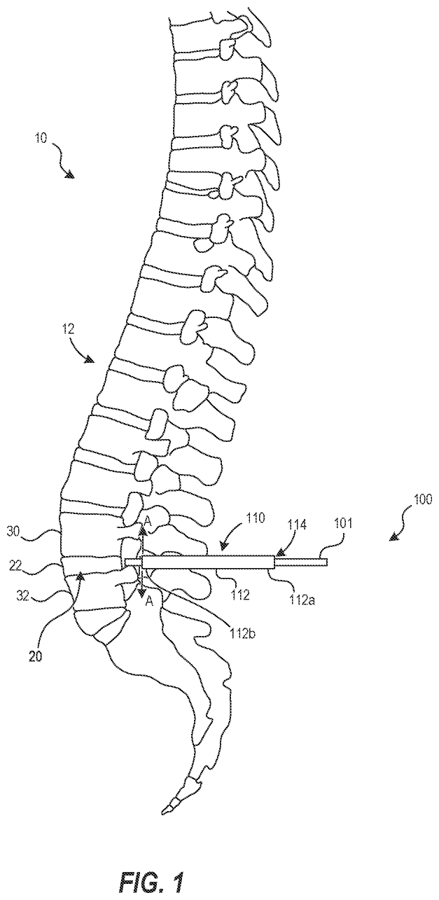

Certain details are set forth in the following description and in FIGS. 1-9 to provide a thorough understanding of various embodiments of the present technology. In other instances, well-known structures, materials, operations, and/or systems often associated with spinal procedures, spine surgical instrument, intervertebral devices, and the like are not shown or described in detail in the following disclosure to avoid unnecessarily obscuring the description of the various embodiments of the technology. Those of ordinary skill in the art will recognize, however, that the present technology can be practiced without one or more of the details set forth herein and/or with other structures, methods, components, and so forth.

With regard to the terms “distal” and “proximal” within this description, unless otherwise specified, the terms can reference a relative position of the portions of a retractor with reference to an operator and/or a location in the patient. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

FIG. 1 is a side view of a spinal surgical system 100 (“system 100”) positioned along a human subject's spine 10 in accordance with embodiment of the present technology. The system 100 can include an instrument holder assembly in the form of an access tool 110 (which can also be referred to as a “access assembly,” an “inserter,” a “cannula,” a “retractor,” an “expandable cannula,” and the like). The access tool 110 can include a cannula or a tubular body 112 (“body 112”) having a first (e.g., proximal) end portion 112a, a second (e.g., distal) end portion 112b opposite the first end portion 112a, and a lumen 114 extending therebetween. The access tool 110 can be configured to provide a user (e.g., a practitioner, a surgeon, a clinician, and the like) with access to one or more of the patient's tissues 12 during a surgical procedure. In the illustrated embodiment, for example, the second end portion 112b of the access tool 110 is positioned proximate an intervertebral space 20 between two vertebral bodies 30, 32 within the subject's spine 10. The access tool 110 can be used to access the intervertebral space 20, or another suitable target location within the patient, via one or more different delivery paths, such as an ALIF path, an OLIF path, an LLIF path, an XLIF path, a TLIF path, and/or any other suitable delivery path.

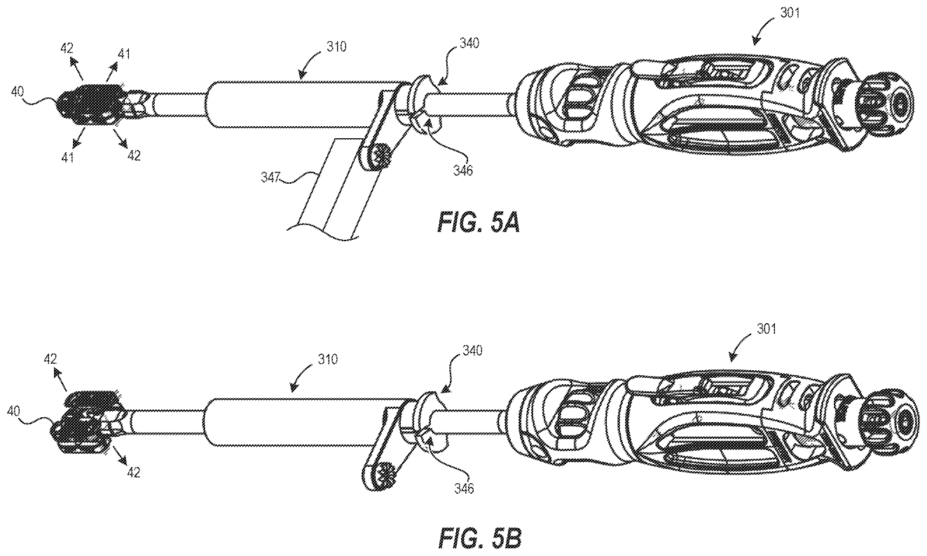

The access tool 110 can be configured to at least partially or fully prevent the intervertebral space 20 from decreasing in size and/or otherwise partially or fully prevent movement of the vertebral bodies 30, 32, for example, after removal of an intervertebral disc 22 positioned within the intervertebral space 20. In some embodiments, for example, the access tool 110 can be expandable for adjusting or increasing the size (e.g., a cross-sectional area, a dimension, a diameter, a width, a height, etc.) of a passage to the intervertebral space 20, or any other suitable target location within the subject. In these and other embodiments, the access tool 110 can be operable to hold instruments used to manipulate (e.g., retract, distract, and the like) the patient's tissues as part of the surgical procedure, or combinations thereof. In at least some embodiments, for example, the access tool 110 can hold an intervertebral spacer delivery instrument, as discussed in connection with FIGS. 5A-5B. In at least some embodiments, the access tool 110 is operable to retract tissue and/or move one or more of the patient's vertebral bodies in the direction indicated by the arrows A in FIG. 1, so as to enlarge the intervertebral space 20 and/or otherwise prepare the intervertebral space 20 to receive an implantable device. Access tools that can operate as retractors are discussed in connection with FIGS. 6A-8C.

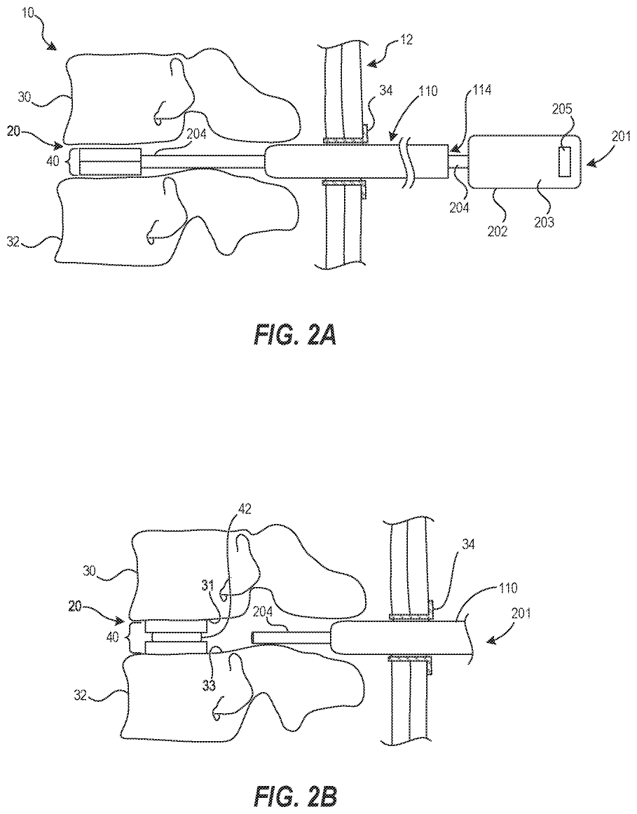

With continued reference to FIG. 1, the system 100 can further include one or more instruments 101. Each of the instruments can be insertable within the subject via the access tool 110, for example, as part of the surgical procedure. In the illustrated embodiment, for example, the instrument 101 is inserted fully through the lumen 114 of the access tool 110 such that a terminus (e.g., a distal terminus) of the instrument is positioned proximate the intervertebral space. At least some of the instruments 101 can be configured to prepare target location within the subject by, for example, moving organs or tissue (e.g., moving nerve tissue), removing tissue (e.g., removing the intervertebral disc 22, removing tissue contributing to stenosis, etc.), preparing vertebral bodies (e.g., roughening or shaping vertebral endplates), or the like. The instrument 101 can be a dilator, an expander, an implant delivery instrument, or other instrument disclosed herein. Additionally, or alternatively, at least some of the instruments 101 can be used to deliver an intervertebral implant or spacer into the intervertebral space 20 through the access tool 110, as described in detail with reference to FIGS. 2A and 2B.

FIGS. 2A and 2B are side views of an intervertebral spacer 40 positioned in the intervertebral space 20 in accordance with embodiments of the present technology. In some embodiments, the intervertebral spacer 40 can be expandable. In the illustrated embodiment, for example, the intervertebral spacer 40 is configured to transition between a first (e.g., delivery, unexpanded, and the like) configuration, shown in FIG. 2A, and a second (e.g., expanded, deployed, and the like) configuration, shown in FIG. 2B. In the second configuration, the intervertebral spacer 40 can be configured to engage and/or support the subject's spine, for example, as part of a surgical procedure to address or treat the subject's spine. In the illustrated embodiment, for example, the intervertebral spacer 40 contacts respective endplates 31, 33 of one or both of the vertebral bodies 30, 32, or any other suitable portion of one or both of the vertebral bodies 30, 32. The intervertebral spacer 40 can be a device (e.g., a dual expansion spacer, an expandable implant, a spacer, etc.) disclosed in U.S. Pat. Nos. 10,105,238; 10,898,340; 6,648,917; 6,562,074; 6,852,129; 6,863,673; 8,628,576; 9,308,099; 10,201,431; 10,105,238; 10,898,340; 10,945,859; and 9,820,788, which are incorporated by reference herein in their entireties.

Referring now to FIG. 2A, the intervertebral spacer 40 can be releasably coupled to an instrument 201 and inserted into the intervertebral space 20 via the lumen 114 of the access tool 110 and/or one or more ports 34 through the patient's tissue 12. The instrument 201 can include at least some aspects that are generally similar in structure and/or function to the instrument 101 of FIG. 1. In the illustrated embodiment, the instrument 201 includes a handle assembly 202, and a delivery assembly 204. The handle assembly 202 can include a grip 203 and one or more control elements 205. The control elements 205 can include one or more dials, levers, triggers, or other movable elements. One or more of the control elements 205 can be operably coupled to the intervertebral spacer 40 and configured to (i) drive the transitioning of the intervertebral spacer 40 between the first and second configurations, and/or (ii) decouple the instrument 201 from the intervertebral spacer 40. In at least some embodiments, for example, the grip 203 and/or at least one of the control elements 205 can be used and/or configured to apply a proximal force to the delivery assembly 204 to decouple the drive assembly 206 from the intervertebral spacer 40. The delivery assembly 204 can be connected to the grip 203 and can include one or more rods, shafts, or other elements used to manipulate and/or operate the intervertebral spacer 40. Referring to FIG. 2B, when the intervertebral spacer 40 is in the second configuration, the delivery instrument 201 can be separated from a connection feature or connection interface 42 (“connection feature 42”) of the intervertebral spacer 40. The connection feature 42 can be releasably coupled to the drive assembly 224 and operable to transition the intervertebral spacer 40 between the first and second configurations. In some embodiments, the connection feature 42 can include a locking member releasably coupled to a proximal end of the drive assembly 206 and insertable into the intervertebral spacer 40. To reposition the intervertebral spacer 40, the instrument 200 can be reconnected to the intervertebral spacer 40 and operated to unlock and collapse the intervertebral spacer 40. The instrument 201 can be an instrument disclosed in U.S. Pat. Nos. 10,105,238; 10,898,340; 6,648,917; 6,562,074; 6,852,129; 6,863,673; 8,628,576; 9,308,099; 10,201,431; 10,105,238; 10,898,340; 10,945,859; and 9,820,788, which are incorporated by reference herein in their entireties.

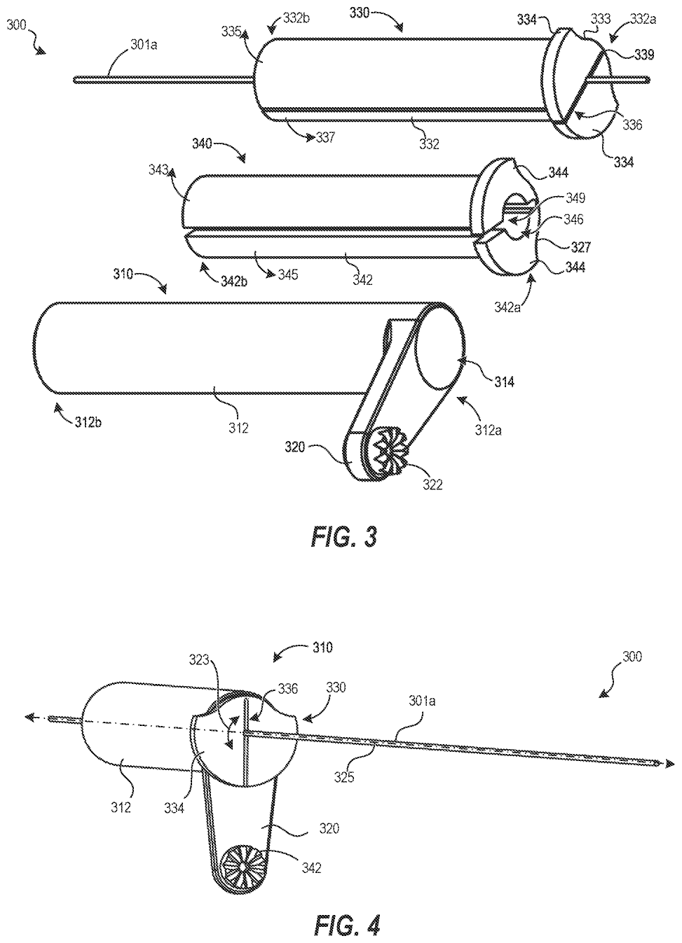

FIGS. 3-5B are perspective views of an instrument holder assembly or system 300 (“system 300”) configured in accordance with embodiments of the present technology. Referring to FIG. 3, the system 300 is configured to position a guidewire 301a and then hold one or more additional instruments. The system 300 can include a cannula assembly 310 (“cannula assembly 310” or “cannula 310”), and one or more guide or holder assemblies 330, 340. At least some aspects of the cannula 310 can be generally similar or identical in structure and/or function to one or more aspects of the access tool 110 of FIGS. 1-2B. In the illustrated embodiment, for example, the cannula assembly 310 includes a working tubular body 312 having a first end portion 312a and a second end portion 312b, and a lumen 314 extending therebetween. The body 312 can have a generally cylindrical cross section, elliptical cross section, polygonal cross section, or other suitable cross-sectional shape. The lumen 314 can be sized such that one or more instruments 301 can be positioned within the lumen 314, for example, for insertion in a subject via the retractor 310 during a surgical procedure.

The cannula assembly 310 can further include one or more mounting or attachment arms 320. In the illustrated embodiment the retractor assembly 310 includes one attachment arm 320. In other embodiments, the retractor assembly 310 can include more attachment arms 340, such as at least, two, three, or any other suitable number of attachment arms 320. The attachment arm 320 can be coupled to the body 312 and extend outwardly from the body 312, for example, in a direction generally perpendicular to a longitudinal axis of the body 312. In some embodiments, the attachment arm 320 is detachably coupled to the body 312, such that the attachment element 320 to be coupled to different regions of the body 312 and/or otherwise repositioned relative to the body 312. In other embodiments, the attachment arm 320 is integrally formed with or part of a single-piece assembly that includes the body 312. In these and other embodiments, the attachment arm 320 can include one or more coupling features 322 (e.g., threaded members, clamping members, etc.) configured for attaching the retractor assembly 310 to one or more platforms, surgical arms, stationary attachment points, or the like, to, for example, hold the retractor assembly 310 in a generally or substantially stationary position and/or orientation relative to a subject (e.g., a target implant location within the subject) during a surgical procedure. The position, number, and configurations of the coupling features 322 can be selected based on the surgical procedure to be performed.

The illustrated system 300 includes two guide assemblies 330, 340, e.g., a first guide assembly 330 and a second guide assembly 340. In other embodiments, the system 100 can include more or fewer guide assemblies, such as at least one, three, four, or any other suitable guide assemblies. As described in greater detail below, each of the guide assemblies can be configured to be at least partially insertable into the cannula 310 and to correspond to one or more instruments (e.g., guidewires, trocars, dilators, spacer delivery tools, or insertion guides, and the like) used during a surgical procedure, such that a single cannula 310 can be used to introduce a plurality of instruments into a subject. In at least some embodiments, for example, the system 300 includes a kit with an instrument positioning assembly, such as the cannula 310, a set of guide assemblies, and a plurality of surgical instruments and respective guide assemblies.

The first guide assembly 330 can include a split sleeve or tubular body 332 having a first end portion 332a and a second end portion 332b opposite the first end portion 332a. The guide body 332 can be made, in whole or in part, of one or more metals, plastics, composite, or other suitable material for engaging (e.g., slidably engaging, etc.), holding, and/or contacting instruments and/or other elements. In at least some embodiments, the body 332 can be radio translucent. The guide body 332 can be configured such that the first guide assembly 330 can be positioned/inserted within the lumen 314 of the retractor 310 and/or rotated relative to the retractor 310 when positioned/inserted within the lumen 314. In some embodiments, the first guide assembly 330 can further include a clamp 333 configured to receive and hold the guidewire 301a. To insert the guidewire, a user can pull part opposing sections of the guide body 332, as indicated by arrows 335, 337. The sections can be biased toward one another by a hinge 339 (e.g., a living hinge, a mechanical hinge, etc.) to hold the guidewire 301a. A user can repeatedly open and close the first guide assembly 330 to reposition the guidewire 301a before, during, and/or after insertion of the guidewire 301a into the patient.

The clamp 333 can include one or more flanges or tabs 334 positioned proximate the first end portion 332a of the guide body 332 and extending outwardly from the guide body 332 such that, when the guide assembly 330 is positioned within the lumen 314 and moved distally, the tabs 334 can contact the first end portion 312a and prevent further distal movement of the first guide assembly 330 relative to the retractor assembly. In other embodiments, the guide assembly 330 can include any other suitable number of tabs 334 and/or tabs in any other suitable position and/or configuration.

The first guide assembly 330 can further include a first guide or receiving feature 336. In the illustrated embodiment, the first receiving feature 336 is an expandable opening slot or a gap positioned at least partially between the tabs 334. In other embodiments, the first guide feature 336 can include a circular, triangular, square, rectangular, or any other suitably shaped opening or aperture. In these and other embodiments, the first receiving feature 336 can be at least partially aligned with a longitudinal axis of the guide body 332 and can define a lumen or guide passageway extending therethrough. When the guidewire 301a is positioned in the first receiving feature 336, the inner edges of the tabs 334 can hold the guidewire 301a stationary. In these and other embodiments, the first receiving feature 336 can extend (e.g., radially outward) through the guide body 332, for example, forming a longitudinal slot through the guide body 332, as shown in FIG. 3. The first receiving feature 336 can be configured to keep or hold one or more instruments in position (e.g., generally centered or at another location) relative to the longitudinal axis of the guide body 332. In the illustrated embodiment, for example, a guide wire 301a is inserted through the first guide assembly 330 via the first receiving feature 336. The guide wire 301a can be moved, rotated, angled, and/or otherwise repositioned when inserted through the first receiving feature 336, for example. In the illustrated embodiment, for example the guide wire 301a can slide, rotate, or be angled relative to a longitudinal axis of the body 332 while positioned within the slot-shaped first receiving feature 336. In these and other embodiments, the position, size, and/or configuration of the first receiving feature 336 can be selected based on the desired positioning of other instruments (e.g., implants, anchors, etc.) delivered via the first guide assembly 330 and/or the retractor assembly 310. Additionally, or alternatively, one or more of the tabs 334 can be movably coupled to guide assembly 330 via one or more hinges, flex features, or joints, such that one or more of the tabs 334 can move or pivot relative to the guide body 332 to increase and/or decrease a size of the first guide feature 336.

The second guide assembly 340 can be configured to receive and hold one or more instruments or tools. The second guide assembly 340 can include a guide body 342 having a first end portion 342a and a second end portion 342b opposite the first end portion 342a. The guide body 342 can be configured such that the second guide assembly 340 can be positioned/inserted within the lumen 314 of the cannula 310 and/or rotated relative to the cannula 310 when positioned/inserted within the lumen 314. When the second guide assembly 340 is inserted within the lumen 314, the second guide assembly 340 can be snugly hold an instrument centered in the lumen 314 of the cannula. The In some embodiments, the second guide assembly 340 can further include one or more flanges or tabs 344, each of which can be generally similar or identical in structure and/or function to the tabs 334 of the first guide assembly 330. A user can grip and pull the tabs 344 outwardly to open the second guide assembly 340. For example, the user can grip and pull the tabs 344 outwardly to open the second guide assembly 340, as indicated by arrows 343, 345. Additionally, or alternatively, insertion of the instrument can open the second guide assembly 340. The tabs 344 can be biased toward one another by a hinge (e.g., a living hinge, a mechanical hinge, etc.) configured to hold the instrument. A user can repeatedly open and close the second guide assembly 340 to reposition the instrument.

The second guide assembly 340 can further include a second guide or receiving feature 346, which can be generally similar or identical in structure and/or function to the first receiving feature 336 of the first guide assembly 330. In the illustrated embodiment, the second receiving feature 346 includes a circular opening positioned at least partially between the tabs 334 and configured to slidably receive the instrument 301. Additionally, or alternatively, the second receiving feature can include an instrument-receiving lumen 349 having a generally circular cross section extends distally from the circular opening and can be configured to receive decompression tools, implantation instruments, imaging equipment, or the like. In these and other embodiments, the position, size, and/or configuration of the second guide feature 346 can be selected based on the desired positioning of other instruments (e.g., implants, anchors, etc.) delivered via the second guide assembly 340 and/or the retractor assembly 310.

During a surgical procedure, one or more of the guide assemblies 330, 340 can be positioned within the cannula 310 to introduce the corresponding instrument(s) into the subject. For example, a user can position the first guide assembly 330 within the cannula 310 to allow the user to navigate the guidewire 301a at least proximate to a target location (e.g., an intervertebral space and/or target implant location) while the guidewire 301a is held/stabilized at least partially by the first guide assembly 330, as shown in FIG. 4. As shown in FIG. 4, the guide 330 can be rotated relative to the cannula 310 to, for example, rotate the guidewire 301a, eccentrically position the guidewire 301a (e.g., when the guidewire 301a is positioned off-center with respect to the lumen 314 (FIG. 3)), etc. For example, the first guide 330 can be rotated, as indicated by arrows 323, relative to longitudinal axis 325 (FIG. 4) of the lumen 314 or cannula body 312. The tabs 334 can have shoulders 327 extending radially outward past the tubular body 312. A user can press upon the shoulder 327 to rotate the first guide 330. In some embodiments, the first guide 330 has shoulders 327 extending from opposing sides of the tubular body 332 for convenient rotation in opposite directions.

In some embodiments, the user can couple the cannula 310 to one or more supporting/stabilizing structures via the coupling feature 322 of the attachment 320, for example, to further stabilize the guidewire 301a during insertion. Additionally, or alternatively, the user can remove the first guide assembly 330 and/or guide wire 301 and insert the second guide assembly 340 within the cannula 310.

FIG. 5A shows the second guide assembly 340 holding the instrument 301. The cannula 310 and second guide assembly 340, for example, allow the user to position the spacer delivery instrument 301 at the target location identified using the guidewire 301a. In some embodiments, the user can partially or fully expand the spacer 40 while the spacer is positioned at least partially or fully within the retractor 310, for example, to test the operation of the spacer 40 and/or confirm that the spacer 40 is properly sized prior to implantation. Additionally, or alternatively, the second guide assembly 340 can be pulled proximally and removed from the cannula 310 to provide increased range of motion of the instrument 301 relative to/within the cannula 310. The second guide assembly 340, or another guide assembly, can then be placed over the shaft of the instrument 301 (e.g., by hinging the second guide assembly 340 open, as shown by the arrows 343, 345 in FIG. 3) and then slide into the cannula 310. Any number of guide assembly can be inserted into the cannula 310 to sequentially position one or more instruments in the cannula 310.

FIGS. 5A and 5B show the instrument 301 releasably and/or operably coupled to the intervertebral spacer 40 with the intervertebral spacer 40 in different configurations. In FIG. 5A, the intervertebral spacer 40 is shown in an unexpanded configuration. In Figure the intervertebral spacer 40 is shown in a first (e.g., horizontally, laterally, and the like) expanded configuration, in which the intervertebral spacer 40 has been expanded in the direction indicated by arrows 41. Additionally, or alternatively, the intervertebral spacer 40 can be configured to expand to a second (e.g., vertically) expanded configuration, in which the intervertebral spacer 40 is expanded in the direction indicated by arrows 42. Additional details regarding intervertebral spacers can be found in such as described in U.S. Pat. No. 10,105,238, the entirety of which is incorporated by reference herein. The second guide assembly 340 can hold the instrument 301 centered in the cannula 310 without inhibiting movement of internal components of the instrument 301. To grip the instrument 301 securely to the cannula 310, the second guide assembly 340 can be made, in whole or in part, of a compressible material, rubber, silicon, or another compressible material. To slidably hold the instrument 301 within the cannula 310, the second guide assembly 340 can be made, in whole or in part, of a rigid plastic, metal, or another material with smooth contact surfaces along which the instrument 301 can slide. In these and other embodiments, the cannula 310 can be used to deliver instruments without using any guides for increase maneuverability of the instruments.

FIGS. 6A and 6B are perspective views of another cannula 610 configured in accordance with embodiments of the present technology. At least some aspects of the cannula 610 can be generally similar or identical in structure and/or function to one or more aspects of the cannula 110 of FIGS. 1-2B and/or the cannula assembly 310 of FIGS. 3-5B, with like reference numbers indicating generally similar or identical aspects. The cannula 610 can include a tapered body 612. The degree of taper (e.g., radial taper) of the body 612 can be equal to or greater than about 2 degrees, 5 degrees, 10 degrees, 15 degrees, or 20 degrees. In the illustrated embodiment, the body 612 is tapered distally such that the body 612 has a first dimension (e.g., diameter, width, and the like) at the first or proximal end portion 612a and a second dimension is less than the first dimension at a second or distal end portion 612b. In other embodiments, the body 612 can be tapered proximally. Additionally, or alternatively, the body 612 can be tapered along a section, multiple sections, or most of the length of the body 612. In these and other embodiments, the body 612 can include a sequence of one or more radially tapered and/or radially flared portions, or any other suitable profile along its longitudinal length or a portion thereof.

The cannula 610 can be used with other components discussed herein. In some embodiments, an instrument guide (e.g., first or second guides 330, 340 of FIGS. 3-5B) can have configurations complementary to the cannula 610. An inner lumen 614 of the cannula 610 can be tapered distally to match the exterior taper. The outer taper of the cannula 610 can similar or identical to the taper of the lumen 614 such that the body 612 limits the insertion depth of the guides. In some embodiments, the guides can have substantially uniform diameters along their lengths such that the body 612 compresses the guides as the guides are advanced distally into the cannula 610, thereby at least partially collapsing the guides. This allows a user to control the compressive or gripping forces applied to instruments within the cannula 610.

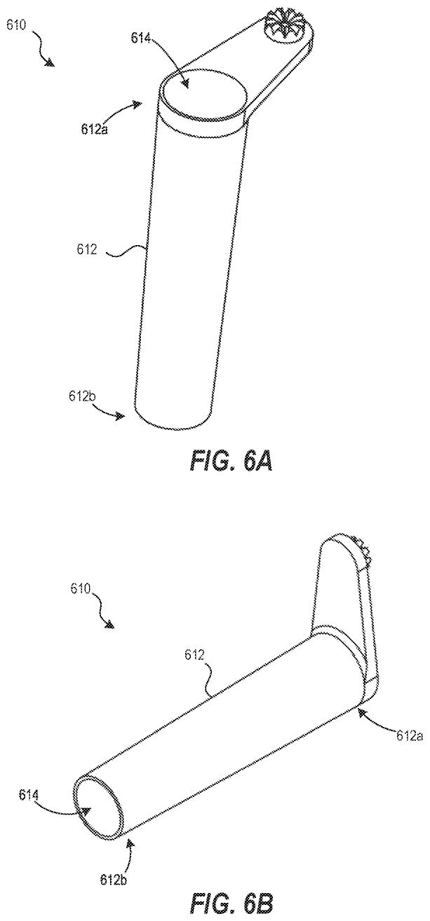

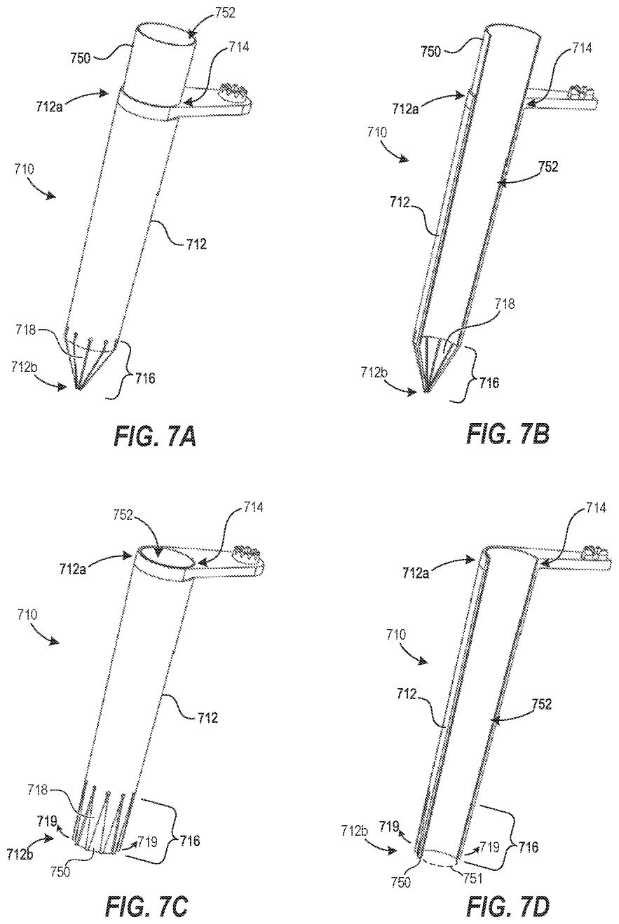

The cannulas and cannula assemblies of FIGS. 1-6B can include features discussed in connection FIGS. 7A-8C. For example, the cannula 110 of FIGS. 1-2B, the cannula assembly 310 of FIGS. 3-5B, and/or the cannula assembly 610 of FIGS. 6A and 6B can include expandable regions, insertable expanders, deployable portions (e.g., longitudinally extending deployable member, panels, etc.), and/or other features disclosed herein. Additionally, the components discussed in connection with FIGS. 1-6B can be used with the embodiments discussed in connection with FIGS. 7A-8C. For example, guides can be used to position instruments in the retractors and retractor assemblies of FIGS. 7A-8C.

FIGS. 7A-7D are perspective views of a retractor assembly 710 configured in accordance with embodiments of the present technology. Specifically, FIGS. 7A and 7B are perspective and cross-sectional perspective views, respectively, of the retractor assembly 710 in a first (e.g., unexpanded) configuration, and FIGS. 7C and 7D are perspective and cross-sectional perspective views, respectively, of the retractor assembly 710 in a second (e.g., expanded) configuration. At least some aspects of the retractor assembly 710 can be generally similar or identical in structure and/or function to one or more aspects of the cannula 110 of FIGS. 1-2B, the cannula assembly 310 of FIGS. 3-5B, and/or the cannula assembly 610 of FIGS. 6A and 6B, with like reference numbers indicating generally similar or identical aspects.

The body 712 of the retractor 710 can include an expandable region 716. In the illustrated embodiment, the expandable region 716 is positioned proximate the second end portion 712b of the body 712, such that expanding the expandable region 716 can produce a corresponding expansion of the second end portion 712b of the body 712. In other embodiments, the expandable region 716 can be positioned proximate the first end portion 712a, between the first and second end portions 712a, 712b or at any other suitable position. In some embodiments, the entire distal end portion 712b and/or the entire body 712 can be expandable/stretchable. At least a portion of the expandable region 716 can be made of one or more metals (e.g., nitinol, steel, aluminum, alloys, etc.), plastics, stretchable materials, webbing (e.g., webbing extending between deployable rigid arms or expansion members discussed below), and/or other materials capable of deformation or expansion. In the illustrated embodiment the body 712 is generally cylindrical. In other embodiments, the body 712 can generally be conical, for example, tapered radially inward as described in detail regarding FIGS. 6A and 6B.

The expandable region 716 includes one or more arms or expansion members 718 configured to bend or deflect relative to a longitudinal axis of body 712. In at least some embodiments, the arms 718 can be biased in a first (e.g., radially inward) direction, as shown in FIGS. 7A and 7B, and can be bent or deflected in a second (e.g., radially outward) direction, as shown in FIGS. 7C and 7D. Additionally, or alternatively, the expandable region 716 can include a plurality of slots, expansion features, or other components that are evenly or unevenly circumferentially spaced apart about the body 712. In these and other embodiments, the length, configuration, and/or spacing of the arms 718 can be selected based on the desired amount of expansion.

In the illustrated embodiment, an insertable expander 750 can be inserted into and moved distally through the lumen 714 to bend or deflect the arms 718 and expands the distal end portion 712b of the body 712, as indicated by the arrows 719 in FIGS. 7C and 7D. The expander 750 can be hollow and define a lumen 752, such that one or more instruments can be positioned with the expander 750. In the illustrated embodiment, the expander 750 has a length equal to the length of the body 712 such that, when the expander 750 is fully inserted within the body 712, the expander 750 extends fully between the first and second end portions 712a, 712b. In other embodiments, the expander 750 can have a length less or greater than the length of the body 712. Additionally, or alternatively, in some embodiments a plurality of expanders 750 can be inserted within the lumen 714 to cause expansion of the expandable region 716. In these and other embodiments, the number, size, and/or the configuration of the expanders 750 can be selected based on the desired amount or degree of expansion to be caused when inserted.

In a surgical procedure, the retractor 710 can be inserted into a patient. Based on visualization, one or more expanders 750 can be selected and then inserted into the lumen 714. The configuration (e.g., cross-sectional shape, cross-sectional dimensions, etc.) of each expander 750 can be selected based on the desired working space. For example, a physician can select one or more expanders 750 having a cross-sectional area and/or shape that matches a desired cross-sectional area and/or shape of a passage through which instruments will be moved. In some embodiments, the unexpanded diameter (FIG. 7A) of the expandable region 716 can be about 2 millimeters, 3 millimeters, 4 millimeters, 5 millimeters, or other diameters. The expanded diameter (FIG. 7b) of the expandable region 716 can be, for example, about 10 millimeters, 15 millimeters, 16 millimeters, 17 millimeters, 18 millimeters, 19 millimeters, 20 millimeters, 25 millimeters, etc. In some embodiments, the expanded dimension of the expandable region 716 can be equal to or greater than 2 times, 3 times, 4 times, or 5 times the starting or unexpanded dimension of the expandable region 716.

The collapsed or unexpanded configuration, expanded configuration, and means for expansion of expandable region 716 can be selected based on the procedure. Advantageously, the expandable region 716 can be expanded at a location at or proximate to a surgical site to define a working envelop 751 (illustrated in dashed line in FIG. 7D) inside the patient. In some procedures, for example, the retractor 710 can be used in the procedure of FIGS. 2A and 2B. The expandable region 716 can expand outwardly against the spinous processes of the vertebrae 30, 32 to expand the intervertebral space 20. The unexpanded portion of the retractor 710 can sealingly engage the port 34. A series of insertable expanders 750 with conical, flared, eccentrically shaped distal portions can be inserted to retractor 710 to move the expandable region 716 between multiple configurations.

FIGS. 8A-8C are perspective views of another retractor assembly 810 configured in accordance with embodiments of the present technology. FIG. 8A illustrates the retractor assembly 810 in a first (e.g., unexpanded) configuration, and FIGS. 8B and 8C illustrate retractor assembly 810 in a second (e.g., expanded) configuration. At least some aspects of the retractor assembly 810 can be generally similar or identical in structure and/or function to one or more aspects of the retractor 110 of FIGS. 1-2B, the retractor assembly 310 of FIGS. 3-5B, the retractor assembly 610 of FIGS. 6A and 6B, and/or the retractor assembly 710, with like reference numbers indicating generally similar or identical aspects.

The retractor 810 includes a body 812 comprising a platform 860 and one or more longitudinally extending members 870 (one identified) that define a generally cylindrical passageway or lumen 814 through the retractor 810 and within which one or more instruments can be inserted. In some embodiments, the platform 860 can include attachment features 842 for coupling to attachment points, arms, fixation elements, or the like. The body 812 and/or the members 870 can include a radially taper, for example, a radially inward taper away from the platform and/or toward the distal end portion 812b. The number, length, and configuration of the members 870 can be selected based on the desired amount of expansion, ability to displace tissue, or the like.

In some embodiments, the members 870 can be hingedly, pivotally, slidably, or otherwise movably coupled to the platform 860. For example, the members 870 can have driven portions 872 (one identified) retained by a slot or receiving feature 862 of the platform 860. The driven portions 872 can include a driven slot 874 operably coupled to a drive feature 864 (linearly movable as indicated by arrow 873) of the platform 860. In the illustrated embodiment the drive feature 864 includes a rotatably gear and the driven slot 874 includes corresponding gear teeth, such that rotary movement of the drive feature 864 can cause linear movement of the driven portions 872 within the receiving feature 862 and move the members 870 relative to the platform 860, as shown by arrows 819 in FIG. 8B, and thereby adjust the dimensions of the lumen 814. Additionally, or alternatively, one or more guidewires, implantable devices, expanders, drivers, and/or instruments can be moved distally through the retractor 810, as indicated by arrow 815, along a delivery axis or path 816 to manually push apart the members 870, thereby expanding the lumen 814. For example, a guide 840 (shown in dashed line in FIG. 8C) can be inserted to the retractor 810. The description of the guides of FIGS. 3-5B applies to the guide 840. In these and other embodiments, the configuration, components, and functionality of the platform 860 can be selected based on the desired fixation features to be used during surgery.

The retractors and components discussed herein can be configured for use with instruments, implants, devices, or other components disclosed in the incorporated by reference applications and patents. Additionally, retractors can be selected based on the anatomical features of the patient. For example, the access tools (e.g., cannulas/retractors) 310, 410, 510, and/or 610 of FIGS. 4-6B, respectively, can be used to access working spaces which do not need to be enlarged after retractor insertion. The retractors 710, 810 of FIGS. 7A-8B, respectively, can be used to create an enlarged working space along the spine.

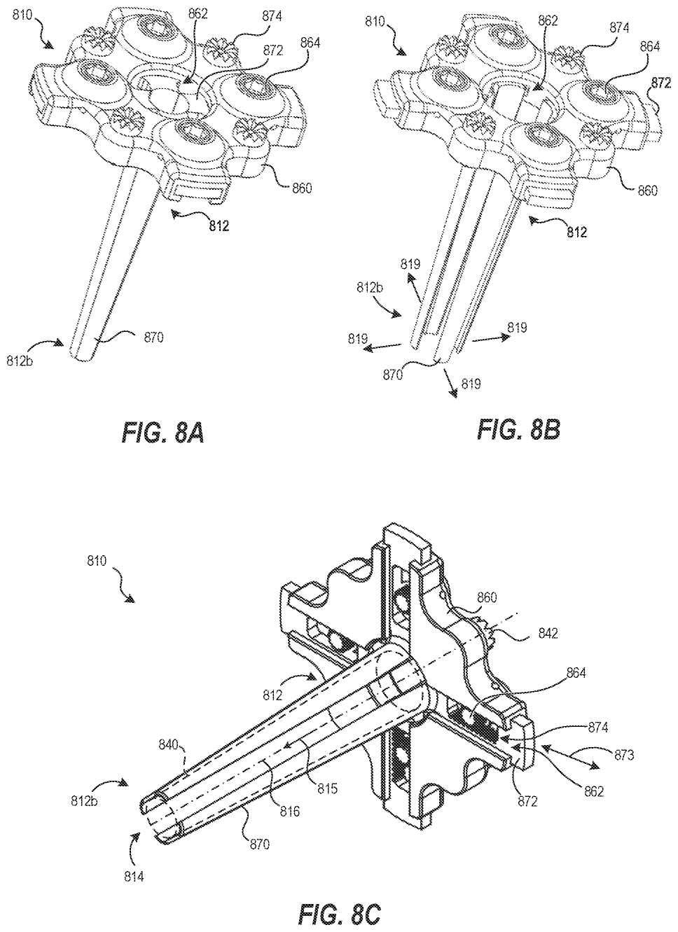

Referring to FIG. 9, an instrument or tool set 901 is depicted which may be used to implant the intervertebral spacer 40 and/or fill the spacer with bone graft material in situ. One or more of the tools 901 can be used in conjunction with at least some of the assemblies, cannulas, and retractors described herein. For example, although certain aspects of the tool set 901 are described with reference to the cannula assembly 310, one or more of the other cannula/retractors described herein can also be used with the tool set 901. The number of components, configuration, and configuration of the access tools can be selected based on the instrument set 901. In some embodiments, a pre-assembled kit can include set of access tools and the instrument set 901 configured to match the access tools. The set of access tools can include cannula assemblies, cannulas, and retractors. A user can select the number and configuration of access tools to perform a mono-portal procedure, multi-portal procedure, etc.

With continued reference to FIG. 9, the instrument set 901 can include a modular inserter instrument 910, a draw bar 920, a graft funnel 930, a tamp 940, and/or a driver 950. The intervertebral spacer 40 may be rigidly mounted to the distal end of the inserter instrument 910. Each of the draw bar 920, graft funnel 930, tamp 940, and driver 950 may be inserted partially through the inserter instrument 910 to perform various functions with the mounted interbody device.

The inserter instrument 910 includes a handle 912, an actuator which may be an inserter knob 914, and a cannulated inserter shaft 916. The shaft 916 can be dimensioned to fit within guides (e.g., as shown in FIGS. 5A and 5B) to allow the inserter instrument 910 to slide along guides to maintain a delivery trajectory while being advanced to an implantation site. For example, referring to FIGS. 5A and 5B, the cannula assembly 310 can securely hold the instrument 301 while a user operates components of the instrument 301 to, for example, deploy an implant. Referring again to FIG. 9, the inserter knob 914 includes a threaded receptacle 913. At least one cutout 917 may be formed into the shaft 916. An attachment port 918 is formed on the distal end of the inserter shaft 916. At least one indicator 915 may be present on the instrument. The inserter instrument 910 further includes first and second levers 911a, 911b connected to first and second jaws 919a, 919b via a pair of control bars 916a, 916b which are received in grooves on the sides of the inserter shaft 916. The control bars 916a, 916b with the first and second levers 911a, 911b may both actuate the first and second jaws 919a, 919b. At the distal end of the inserter shaft 916, the instrument includes a working end with an attachment or gripping mechanism for gripping, rigidly holding, and releasing the intervertebral spacer 40. Gripping mechanism can include the first and second jaws 919a, 919b. The second jaw 919b may be a mirror image of the first jaw. Each jaw 914, 919b is pivotably attached to the distal end of the inserter shaft at the attachment port 918, and each jaw 914, 919b is pivotably attached to the distal end of a control bar 916a, 916b. When levers 911a, 911b are lifted away from the handle 912, jaws 919a, 919b pivot outward from the attachment port 918. When levers 911a, 911b are moved toward the handle 912, jaws 919a, 919b pivot toward the attachment port 918 and can grip an implant such as the spacer 40.

The draw bar 920 can be inserted through inserter instrument 910 and actuated to move the intervertebral spacer 40 and/or any of the other interbody devices disclosed herein between the compact and expanded configurations. The draw bar 920 includes a distally located threaded tip 922, a draw bar shaft 924, a threaded receptacle portion 926 at a proximal end, and a draw bar knob 928. The draw bar shaft 924 may include stepped portions. The threaded tip 922 and receptacle portion 926 may have equal pitch threads. In use, the draw bar 920 is inserted into the inserter handle 910 and shaft 916, with tip 922 extending into and threadably engaging the intervertebral spacer 40 as the threaded receptacle portion 926 enters threaded receptacle 913 of the knob 914. The draw bar 920 is rotated so that threaded tip 922 fully threadably engages the intervertebral spacer 40 simultaneously with threaded receptacle portion 926 fully engages threaded receptacle 913. The threads of tip 922, intervertebral spacer 40, receptacle portion 926, and receptacle 913 are all rotationally oriented for simultaneous threading, and may be rotationally oriented for simultaneous initial engagement of tip 922 in the intervertebral spacer 40 and receptacle portion 926 in receptacle 913. Draw bar knob 928 can prevent over-insertion of the draw bar 920 into the instrument 910 and intervertebral spacer 40. To expand the intervertebral spacer 40, inserter knob 914 is rotated counterclockwise about draw bar 920 to feed draw bar 920 proximally, and thus expand intervertebral spacer 40, such as described in U.S. Pat. No. 10,105,238, the entirety of which is incorporated by reference herein. The cannula assembly 310 can hold the inserter handle 910 stationary (e.g., rotationally fixed, translationally fixed, etc.) relative to the cannula/retractor, a support or frame 347 (Figure to which the cannula assembly 310 is coupled, etc., such that the cannula assembly 310 can inhibit, limit, or substantially prevent unwanted displacement, rotation, etc. of the implant before, during, and/or after expansion.

Once the intervertebral spacer 40 is expanded as desired, draw bar knob 928 may be rotated counterclockwise to disengage from the intervertebral spacer 40 and threaded receptacle 913, and draw bar 920 may be withdrawn from the intervertebral spacer and the inserter instrument 910. The cannula assembly 310 (FIGS. 3-5B) can hold the instrument 910 to limit or prevent movement of the intervertebral spacer 40 relative to the patient during the disengagement process.

In another method of use, draw bar 920 may be used to urge the intervertebral spacer 40 from the expanded to the compact configuration, and to remove the intervertebral spacer 40 from its implanted location. Inserter instrument 910 may be engaged with intervertebral spacer 40 as described above, with jaws 919a, 919b gripping intervertebral spacer 40 while the cannula assembly 310, or any other suitable cannula and/or retractor described herein, holds the instrument 910 in alignment with the spacer 40. Draw bar 920 may be inserted into and engaged with instrument 910 and intervertebral spacer 40 as described previously. Inserter knob 914 may then be rotated clockwise to urge draw bar 920 distally, thus transforming the intervertebral spacer 40 from the expanded configuration. Inserter instrument 910 may then be pulled proximally to remove the intervertebral spacer from its implanted location.

The graft funnel 930 is insertable through inserter instrument 910 held by the cannula assembly 310, or any other suitable cannula and/or retractor described herein, to provide a passageway for packing bone graft or other material in and around an interbody device. Graft funnel 930 includes a funnel shaft 932 which is cannulated and has a distal shaft opening 934, a threaded receptacle portion 936 at a proximal end, a funnel neck 937 and a funnel head 938. In a method of use, graft funnel 930 is inserted into inserter instrument 910 with funnel shaft 932 extending through inserter shaft 916, and shaft opening 934 abutting attachment port 918. Threaded receptacle portion 936 engages with threaded receptacle 923 to hold the graft funnel 930 in its inserted position. Funnel neck 937 may prevent over-insertion of the graft funnel 930 into the instrument and interbody device. The inner diameters of the funnel shaft 932 can provide a smooth, uninterrupted path for graft material, and precluding any pockets or other inclusions where graft material could potentially hang up or be lost. Bone graft material is then fed into funnel head 938, through funnel shaft 932, attachment port 918, and deposited in an interior region of the intervertebral spacer 40.

With continued reference to FIG. 9, the tamp 940 is insertable through the graft funnel 930 to push and/or pack the bone graft material. The tamp 940 includes a handle 942, and a tamp shaft 944 having a distal tip 946. In the embodiment shown, distal tip 946 is concave. In a method of use, the tamp 940 is inserted into the graft funnel 930 after placement of bone graft material in the funnel 930. The tamp shaft 944 is coaxially received in the cannulated funnel shaft 932, and the distal tip 946 pushes the graft material through the funnel shaft 932, attachment port 918, and into the interior of the intervertebral spacer 40. By extending all the way into the intervertebral spacer 40, the tamp 940 may minimize graft waste. The concavity of tip 946 minimizes graft insertion forces and collects graft from the interior of the funnel shaft 932 as the tamp 940 is passed through the graft funnel. The outer diameter of the tamp shaft 944 and tip 946 is smaller than the inner diameter of the funnel shaft 932 with just enough clearance to allow movement of the tamp shaft 944 through the funnel shaft 932, but not enough space to permit loss of graft material between the tamp shaft 944 and the funnel shaft 932.

The cannula assembly 310 can be repositioned any number of times to adjust the orientation and position of the inserter instrument 910. When at a desired position, the cannula assembly 310 can be locked at the desired position to hold the inserter instrument 910 at the target orientation for delivering additional graft material. The positioning of tools, instruments, etc. can be performed under direct visualization, fluoroscopy, etc. In some procedures, the graft material may fill the interior region and spill out of and/or across superior and/or inferior surfaces of the intervertebral spacer 40. The graft material may be pre-measured to ensure placement of a desired amount of material, or to calculate the actual amount of material placed. Pre-measured volumes of material can be correlated to each implanted spacer (e.g., multilevel surgeries in which multiple spaces are implanted, multiple spacers at a single level, etc.) cannula assembly 310 and/or inserter instrument 910 positions. This allows a user to plan surgical procedures in improve subject outcomes.

With continued reference to FIG. 9, the driver 950 is insertable through the inserter instrument 910 to drive a screw, such as the locking feature described in PCT App. No. PCT/US22/19706, the entirety of which is incorporated by reference herein, into the intervertebral spacer 40 to lock the intervertebral spacer 40 in the expanded configuration. The driver 950 includes a handle 952, a driver shaft 954, and a distal driver tip 956. The distal driver tip 956 is complementarily shaped to a drive feature on the screw so that when the driver tip 956 is engaged with the screw, rotating the driver 950 drives the screw. In the example shown, driver tip 956 is hexagonal, but other shapes known in the art, including square, triangular, pentagonal, and star, are contemplated. At or adjacent the distal driver tip 956, a retention feature may be present to assist in connection with the screw as the screw is placed and driven. Retention feature may be a ball detent, taper, twist, spring feature, or other retention features known in the art.

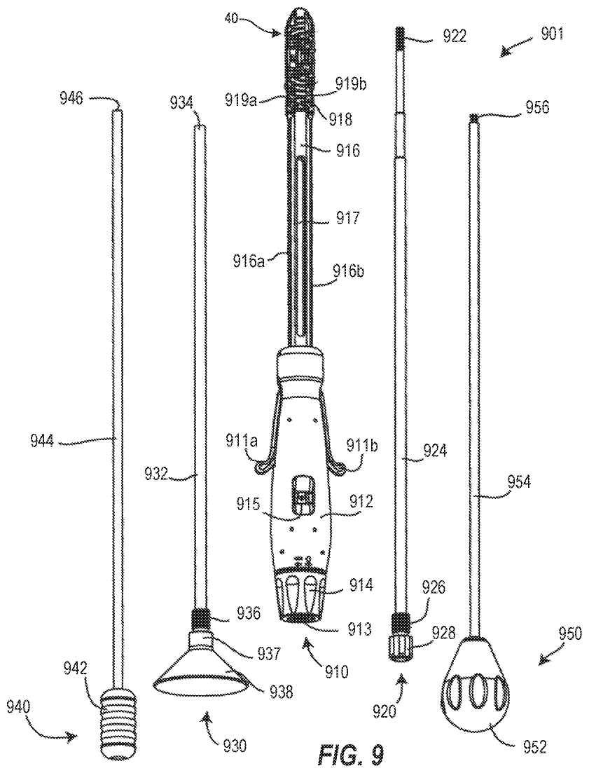

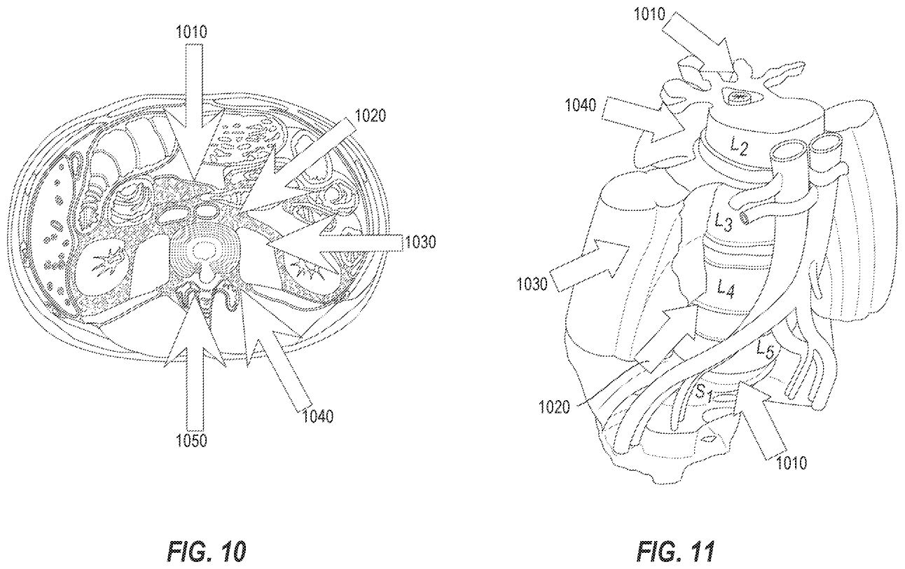

FIG. 10 is a schematic top plan view along the lumbar spine of a human and illustrates example approaches for performing interbody fusion procedures suitable for the systems, devices, and methods described herein with reference to FIGS. 1-9. FIG. 11 is an isometric view of the lumbar spine of FIG. 10. Referring to FIGS. 10 and 11, surgical instruments, such as the access tool, cannulas, and retractors described previously with reference to FIGS. 1-9, can be delivered via different paths, including an anterior lumbar interbody fusion (ALIF) path 1010, an oblique lumbar interbody fusion (OLIF) path 1020, a lateral or extreme lateral lumbar interbody fusion (LLIF or XLIF) path 1030 (e.g., direct lateral), a transforaminal lumbar interbody fusion (TLIF) path 1040, and a posterior lumbar interbody fusion (PLIF) path 1050. The instruments, access tools, and devices disclosed herein can be configured based on the delivery path. For example, a cannula used in an anterior or lateral approach can be dimensioned based on anatomical structures surrounding the implantation site. Retractors can be configured to move tissue, define working spaces/envelopes, etc. without causing adverse events, such as damage or trauma to non-targeted tissue (e.g., nerve tissue, spinal cord, etc.).

With continued reference to FIGS. 10 and 11, the number and configuration of interbody/intervertebral fusion devices can be selected based on the fusion procedure to be performed. In one example TLIF procedure, the transforaminal path 1040 may be employed to implant a single small expandable or non-expandable interbody/intervertebral spacer at the intervertebral space. In one example PLIF procedure, two interbody spacers can be delivered along the posterior path 1050 and implanted at the intervertebral space. The two interbody spacers can cooperate to keep the vertebral bodies at the desired spacing and may be larger than the TLIF spacer. Additionally, multiple interbody spacers can provide lordotic correction by providing support at different heights. In one example LLIF procedure, a single relatively large interbody spacer can be delivered along the lateral path 1030 and implanted to provide asymmetrical support. In one example ALIF procedure, an asymmetric interbody spacer can be delivered along the anterior path 1010 to provide support consistent with lordosis at that portion of the spine. Lateral approaches, transforaminal approaches, and anterior approaches can be used to access the cervical spine, thoracic spine, etc. The number of instruments, configurations of instruments, implants, and surgical techniques can be selected based on the condition to be treated.

The configuration of implants in a delivery state can be selected based on the delivery path in order to avoid trauma to non-targeted tissue, tissue to be preserved, etc. For example, delivery path and/or the distance between non-targeted tissues can be measured (e.g., under direct visualization, using fluoroscopy, etc.) to determine delivery configuration suitable for delivery at an intervertebral space without severing the non-targeted tissue. The expanded configuration can be determined based on the patient's condition. An implant can be selected based on the delivery and expanded configurations. Access tools, instruments, etc. can also be selected based on the dimensions (e.g., width, height, etc.) of the delivery path.

### Examples

Several aspects of the present technology are set forth in the following examples:
1. A cannula system comprising:
a cannula including a proximal end, a distal end, a lumen extending between the proximal and distal ends, and an attachment arm at the proximal end;
a first instrument guide including a first split sleeve configured to be inserted into the lumen of the cannula and to hold a portion of the first instrument spaced apart from a sidewall of the first split sleeve; and
a second instrument guide including a second split body configured to be inserted into the lumen of the cannula and an instrument-receiving lumen, wherein the second split body is configured to hold portions of a second instrument at proximal and distal ends of the cannula when the first instrument is positioned in the instrument-receiving lumen.
2. The cannula system of example 1, wherein the cannula is configured to partially close at least one of the first instrument guide or the second instrument guide such that the at least one of the first instrument guide or the second instrument guide grips the first instrument or second instrument, respectively.
3. The cannula system of example 1 or example 2, wherein the first instrument guide is configured to be held in a collapsed configuration by the cannula to clamp onto the first instrument, wherein the first instrument is a guidewire.
4. The cannula system of any of examples 1-3, wherein the second instrument guide is configured to be held in a collapsed configuration by the cannula to clamp onto a shaft of a second instrument configured to deploy intervertebral spacer.
5. The cannula system of any of examples 1-4, wherein the second instrument guide include a hinge extending along the of the second split body.
6. The cannula system of any of examples 1-5, wherein at least one of the first instrument guide or the second instrument guide is rotatable about a longitudinal axis of the lumen when positioned in the cannula.
7. The cannula system of any of examples 1-6, wherein at least one of the first instrument guide or the second instrument guide has one or more tabs configured to extend radially outwardly past the proximal end of the cannula.
8. The cannula system of any of examples 1-7, wherein at least one of the first instrument guide or the second instrument guide has one or more tabs configured to extend radially outwardly past the proximal end of the cannula.
9. The cannula system of any of examples 1-8, wherein the cannula has an expandable portion at the distal end configured to be radially expanded by the second instrument guide.
10. The cannula system of any of examples 1-9, wherein the cannula includes a tubular body tapered toward the distal end.
11. The cannula system of any of examples 1-10, wherein the cannula includes a tubular body tapered toward the distal end.
12. The cannula system of any of examples 1-11, wherein the cannula is a retractor with a plurality of longitudinally extending members spaced circumferentially about a delivery axis, wherein each of the longitudinally extending members is configured to rotate away from the delivery axis toward an expanded configuration.
13. The cannula system of any of examples 1-12, wherein the second split body has a longitudinally extending gap along its length and is configured to expand or contract to accommodate a shaft of the first instrument.
14. A spinal surgical system, comprising:
a cannula including a tubular body having a first end portion and a second end portion opposite the first end portion, wherein the tubular body defines a cannula lumen extending through the tubular body between the first and second end portions;
a guide assembly including a guide body configured to be insertable at least partially within the cannula lumen, wherein the guide body includes a tab extending from the guide body and positioned to contact the tubular body during insertion of the guide assembly within the cannula lumen, and wherein the guide body further includes a guide feature extending through the guide body; and
a spinal surgical instrument configured to be positioned in the guide feature and held by the guide assembly.
15. The spinal surgical system of example 14 wherein the guide assembly is configured to be rotatable relative to the retractor when at least a portion of the guide assembly is positioned within the cannula lumen.
16. The spinal surgical system of example 14 or example 15, wherein the guide feature includes an inner surface of the guide assembly, and wherein the spinal surgical instrument contracts at least part of the inner surface when the spinal surgical instrument is inserted within the guide feature.
17. The spinal surgical system of any of examples 14-16 wherein the guide feature is configured to allow the spinal surgical instrument to move in at least one direction relative to the guide assembly when at least a portion of the spinal surgical instrument is positioned within the guide feature.
18. The spinal surgical system of example 17 wherein the guide feature is configured to allow the spinal surgical instrument to move longitudinally relative to the guide assembly.
19. The spinal surgical system of example 17 or example 18 wherein the guide feature is configured to allow the spinal surgical instrument to move radially relative to a longitudinal axis of the guide feature.
20. The spinal surgical system of any of examples 17-19 wherein the guide feature is configured to allow the spinal surgical instrument to be angled relative to the longitudinal axis to the guide feature.
21. The spinal surgical system of any of examples 14-20 wherein the guide feature includes a linear slot.
22. The spinal surgical system of any of examples 14-21 wherein the guide feature includes at least one of a circular, triangular, square, and/or rectangular aperture.
23. The spinal surgical system of any of examples 14-22 wherein the spinal surgical instrument includes at least one of a guidewire, a trocar, a cannula, a tissue-removal instrument, and/or an implant-delivery instrument.
24. The spinal surgical system of any of examples 14-23 wherein the guide assembly is a first guide assembly, the guide feature is a first guide feature, and the spinal surgical instrument is a second spinal surgical instrument, the spinal surgical system further comprising a second guide assembly and a second spinal surgical instrument, wherein the second guide assembly includes a second guide feature configured to correspond to the second spinal surgical instrument.
25. The spinal surgical system of example 24 wherein the second spinal surgical instrument is different than the first spinal surgical instrument is different than the second spinal surgical instrument.
26. The spinal surgical system of example 24 or example 25 wherein the second guide assembly has a different configuration than the first guide assembly.
27. The spinal surgical system of any of examples 24-26 wherein the second guide feature has a different configuration than the first guide feature.
28. The spinal surgical system of any of examples 24-27 wherein the second guide assembly includes a second guide body configured to be positioned at least partially within the cannula lumen.
29. The spinal surgical system of any of examples 14-28, wherein the retractor further includes an attachment arm extending from the tubular body.
30. The spinal surgical system of example 29 wherein the attachment arm extends radially outward from a longitudinal axis of the tubular body.
31. The spinal surgical system of example 29 or example 30 wherein the attachment arm includes an attachment feature, the spinal surgical system further comprising a retractor stabilization component, and wherein the attachment feature is configured to releasably couple the retractor to the retractor stabilization component.
32. The spinal surgical system of any of examples 14-31 wherein the tubular body is tapered radially inward.
33. The spinal surgical system of any of examples 14-32 wherein the tubular body includes an expandable region configured to expand radially outward.
34. The spinal surgical system of example 33 wherein the expandable region includes a plurality of expandable members.
35. The spinal surgical system of example 34 wherein individual ones of the plurality of the expandable members are formed in the second end portion of the tubular body.
36. The spinal surgical system of any of examples 33-35, further comprising an expandable insert configured to be insertable at least partially within the cannula lumen to drive expansion of the expandable region.
37. A method of treating a patient's spine, the method comprising:
positioning a distal end of a cannula proximate a target location along the patient's spine;
inserting a tool guide assembly at least partially within a cannula lumen of the cannula; and
positioning a spine surgical instrument proximate the target location, wherein positioning the spine surgical instrument proximate the target implant location includes inserting the spine surgical instrument through a guide feature of the tool guide assembly.
38. The method of example 37 wherein inserting the tool guide assembly at least partially within the cannula lumen includes inserting the tool guide assembly until a tab of the tool guide assembly contacts the cannula.
39. The method of example 37 or example 38 wherein positioning the spine surgical instrument proximate the target location includes changing an orientation of the spine surgical instrument relative to the tool guide assembly.
40. The method of any of examples 37-39 wherein positioning the spine surgical instrument proximate the target implant location includes moving the spine surgical instrument relative to the tool guide assembly.
41. The method of any of examples 37-40 wherein inserting the tool guide assembly includes causing an expandable region of the cannula to expand.
42. The method of any of examples 37-41 wherein the spine surgical instrument includes an expandable intervertebral spacer, the method further comprising actuating the spine surgical instrument to cause the intervertebral spacer to expand.
43. The method of example 42 wherein actuating the spine surgical instrument includes actuating the spine surgical instrument while at least a portion of the expandable intervertebral spacer is positioned within the retractor.
44. The method of any of examples 37-43 wherein the spine surgical instrument is a first spine surgical instrument and tool guide assembly is a first tool guide assembly configured to correspond to the first spine surgical instrument, the method further comprising:
withdrawing the first tool guide assembly from the cannula lumen; and
positioning a second tool guide assembly at least partially within the cannula lumen, wherein the second tool guide assembly is configured to correspond to a second spine surgical instrument.
45. The method of example 44 wherein positioning the second tool guide assembly includes positioning a tool guide assembly having a different configuration than the first tool guide assembly.
46. The method of example 44 or example 45 wherein positioning the second tool guide assembly includes positioning a distal end of the second tool guide assembly proximate the target location.
47. The method of any of examples 44-46 wherein withdrawing the first tool guide assembly includes withdrawing the first spine surgical instrument from the cannula lumen.
48. The method of any of examples 44-47 further comprising positioning the second spine surgical instrument at least partially within a second guide feature of the second tool guide assembly, wherein the second tool feature is configured to correspond to the second spine surgical instrument.
49. The method of example 48 wherein positioning the second spine surgical instrument includes positioning the second spine surgical instrument proximate the target location.
50. The method of example 48 or example 49 wherein positioning the second spine surgical instrument includes positioning a spine surgical instrument different than the first spine surgical instrument.
51. The method of any of examples 37-50 wherein positioning the distal end of the cannula proximate the target location along the patient's spine includes inserting the cannula along a lateral lumbar interbody fusion path or a posterior lumbar interbody fusion path.
52. The method of any of examples 37-50 wherein positioning the distal end of the cannula proximate the target location along the patient's spine includes inserting the cannula along an anterior lumbar interbody fusion path, an oblique lumbar interbody fusion path, or a transforaminal lumbar interbody fusion path.
53. A spine surgical system, comprising:
an expandable intervertebral spacer;
a spine surgical instrument operably coupled to the expandable intervertebral spacer and actuable to transition the intervertebral spacer between an unexpanded configuration to an expanded configuration; and
a retractor including a retractor body, wherein the retractor body defines a retractor lumen configured to contain at least a portion of the expandable intervertebral spacer while the expandable intervertebral spacer transitions between the unexpanded configuration and the expanded configuration.
54. A method of treating a target location along a patient's spine, the method comprising:
positioning an intervertebral device within a retractor;
causing the intervertebral device to transition from an unexpanded configuration and an expanded configuration while the intervertebral device is positioned within the retractor;
causing the intervertebral device to transition from the expanded configuration toward the unexpanded configuration;
advancing the intervertebral device distally through an end of the retractor; and causing the intervertebral device to transition from the unexpanded configuration to the expanded configuration to engage at least a portion of the patient's anatomy proximate the target location.
55. A retractor assembly, comprising:
a retractor body having a first end portion and a second end portion opposite the first end portion;
wherein the retractor body defines a retractor lumen extending through the retractor body between the first and second end portions and configured to receive one or more spine surgical instruments, and
wherein the retractor body includes an expandable region configured to expand in response to the insertion of at least one of the spine surgical instruments.

### CONCLUSION

The disclosed medical devices, instruments, or any of their components can be made of a wide range of materials, including any biologically adaptable or compatible materials. Materials considered acceptable for biological implantation include, but are not limited to, stainless steel, titanium, tantalum, combination metallic alloys, various plastics, polymers, resins, ceramics, biologically absorbable materials and the like. Any assembly or its components can also be entirely or partially made of a shape memory material or other deformable material.

The retractor assemblies disclosed herein can be used with non-expandable devices (e.g., screws, cages, etc.), expandable devices (e.g., expandable implants), or other devices. For example, the retractor assemblies can be used with devices for reducing nerve compression, maintaining height of the spine or spine segment, and/or restoring stability to the spine. The retractor assemblies can also be used in non-medical applications. For example, the retractor assemblies can be used to position and/or implant bolts, screws (e.g., locking screws, bone fixation screws, etc.), or other elements configured to engage implantable devices.

Devices, implants, instruments, methods, and related technologies are disclosed in U.S. Pat. Nos. 10,105,238; 10,898,340; 6,648,917; 6,562,074; 6,852,129; 6,863,673; 8,628,576; 9,308,099; 10,201,431; 10,105,238; 10,898,340; 10,945,859; 9,820,788; U.S. application Ser. Nos. 16/565,403; 16/687,520; 17/125,633; 15/793,950; 16/394,244; 15/970,212; 15/500,969; U.S. Provisional App. Nos. 63/169,799; 63/163,489; 63/163,521; 63/169,804; 63/163,536; 61/442,482; and PCT App. Nos. PCT/US20/49920; PCT/US21/63881; PCT/US22/19706; the entireties of which are hereby incorporated by reference. For example, the systems, instruments, devices, etc., incorporated by reference herein can be incorporated into or used with the technology disclosed herein. The skilled artisan will recognize the interchangeability of various features from different embodiments disclosed herein and incorporated by reference herein. These technologies can be used with, incorporated into, and/or combined with systems, methods, features, and components disclosed herein. All of the applications, publications, and patents cited herein are incorporated by reference in their entireties. Various features of the embodiments disclosed herein may be mixed and matched to provide additional configurations which fall within the scope of the invention. One or more embodiments may be implanted together to provide the precise support and/or correction needed to restore sagittal alignment and balance.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

It should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim in this or any application claiming priority to this application require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment.

The Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as the position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the present technology. In addition, those of ordinary skill in the art will appreciate that further embodiments of the present technology can be practiced without several of the details described below.

While specific embodiments and applications of the present invention have been illustrated and described, it is to be understood that the invention is not limited to the precise configuration and components disclosed herein. Various modifications, changes, and variations which will be apparent to those skilled in the art may be made in the arrangement, operation, and details of the methods and systems of the present invention disclosed herein without departing from the spirit and scope of the invention.

What is claimed is:

1. A cannula system comprising:
a cannula including a proximal end, a distal end, a lumen extending between the proximal and distal ends, and an attachment arm at the proximal end;
a first instrument guide including a first split body configured to be inserted into the lumen of the cannula and to hold a portion of a first instrument spaced apart from a sidewall of the first split body; and
a second instrument guide including a second split body configured to be inserted into the lumen of the cannula and an instrument-receiving lumen, wherein the second split body is configured to hold portions of a second instrument at proximal and distal ends of the cannula when the first instrument is positioned in the instrument-receiving lumen.

2. The cannula system of claim 1, wherein the cannula is configured to partially close at least one of the first instrument guide or the second instrument guide such that the at least one of the first instrument guide or the second instrument guide grips the first instrument or second instrument, respectively.

3. The cannula system of claim 1 wherein the first instrument guide is configured to be held in a collapsed configuration by the cannula to clamp onto the first instrument, wherein the first instrument is a guidewire.

4. The cannula system of claim 1, wherein the second instrument guide is configured to be held in a collapsed configuration by the cannula to clamp onto a shaft of the second instrument configured to deploy intervertebral spacer.

5. The cannula system of claim 1, wherein the second instrument guide include a hinge extending longitudinally along the second split body.

6. The cannula system of claim 1, wherein at least one of the first instrument guide or the second instrument guide is rotatable about a longitudinal axis of the lumen when positioned in the cannula.

7. The cannula system of claim 1, wherein at least one of the first instrument guide or the second instrument guide has one or more tabs configured to extend radially outwardly past the proximal end of the cannula.

8. The cannula system of claim 1, wherein at least one of the first instrument guide or the second instrument guide has one or more tabs configured to extend radially outwardly past the proximal end of the cannula.

9. The cannula system of claim 1, wherein the cannula has an expandable portion at the distal end configured to be radially expanded by the second instrument guide.

10. The cannula system of claim 1, wherein the cannula includes a tubular body tapered toward the distal end.

11. The cannula system of claim 1, wherein the cannula includes a tubular body tapered toward the distal end.

12. The cannula system of claim 1, wherein the cannula is a retractor with a plurality of longitudinally extending members spaced circumferentially about a delivery axis, wherein each of the longitudinally extending members is configured to rotate away from the delivery axis toward an expanded configuration.

13. The cannula system of claim 1, wherein the second split body has a longitudinally extending gap along its length and is configured to expand or contract to accommodate a shaft of the first instrument.

14. A spinal surgical system, comprising:
a cannula including a tubular body having a first end portion and a second end portion opposite the first end portion, wherein the tubular body defines a cannula lumen extending through the tubular body between the first and second end portions;
a guide assembly including a guide body configured to be insertable at least partially within the cannula lumen, wherein the guide body includes a tab extending from the guide body and positioned to contact the tubular body during insertion of the guide assembly within the cannula lumen, and wherein the guide body further includes a guide feature extending through the guide body; and
a spinal surgical instrument configured to be positioned in the guide feature and held by the guide assembly.

15. The spinal surgical system of claim 14 wherein the guide assembly is configured to be rotatable relative to the cannula when at least a portion of the guide assembly is positioned within the cannula lumen.

16. The spinal surgical system of claim 14, wherein the guide feature includes an inner surface of the guide assembly, and wherein the spinal surgical instrument contracts at least part of the inner surface when the spinal surgical instrument is inserted within the guide feature.

17. The spinal surgical system of claim 14 wherein the guide feature is configured to allow the spinal surgical instrument to move in at least one direction relative to the guide assembly when at least a portion of the spinal surgical instrument is positioned within the guide feature.

18. The spinal surgical system of claim 17 wherein the guide feature is configured to allow the spinal surgical instrument to move longitudinally relative to the guide assembly.

19. The spinal surgical system of claim 17 wherein the guide feature is configured to allow the spinal surgical instrument to move radially relative to a longitudinal axis of the guide feature.

20. The spinal surgical system of claim 17 wherein the guide feature is configured to allow the spinal surgical instrument to be angled relative to the longitudinal axis to the guide feature.

21. The spinal surgical system of claim 14 wherein the guide feature includes a linear slot.

22. The spinal surgical system of claim 14 wherein the guide feature includes at least one of a circular, triangular, square, and/or rectangular aperture.

23. The spinal surgical system of claim 14 wherein the spinal surgical instrument includes at least one of a guidewire, a trocar, a cannula, a tissue-removal instrument, and/or an implant-delivery instrument.

24. The spinal surgical system of claim 14 wherein the guide assembly is a first guide assembly, the guide feature is a first guide feature, and the spinal surgical instrument is a first spinal surgical instrument, the spinal surgical system further comprising a second guide assembly and a second spinal surgical instrument, wherein the second guide assembly includes a second guide feature configured to correspond to the second spinal surgical instrument.

25. The spinal surgical system of claim 24 wherein the second spinal surgical instrument is different than the first spinal surgical instrument is different than the second spinal surgical instrument.

26. The spinal surgical system of claim 24 wherein the second guide assembly has a different configuration than the first guide assembly.

27. The spinal surgical system of claim 24 wherein the second guide feature has a different configuration than the first guide feature.

28. The spinal surgical system of claim 24 wherein the second guide assembly includes a second guide body configured to be positioned at least partially within the cannula lumen.

29. The spinal surgical system of claim 14 wherein the cannula further includes an attachment arm extending from the tubular body.

30. The spinal surgical system of claim 29 wherein the attachment arm extends radially outward from a longitudinal axis of the tubular body.

31. The spinal surgical system of claim 29 wherein the attachment arm includes an attachment feature, the spinal surgical system further comprising a cannula stabilization component, and wherein the attachment feature is configured to releasably couple the cannula to the cannula stabilization component.

32. The spinal surgical system of claim 14 wherein the tubular body is tapered radially inward.

33. The spinal surgical system of claim 14 wherein the tubular body includes an expandable region configured to expand radially outward.

34. The spinal surgical system of claim 33 wherein the expandable region includes a plurality of expandable members.

35. The spinal surgical system of claim 34 wherein individual ones of the plurality of the expandable members are formed in the second end portion of the tubular body.

36. The spinal surgical system of claim 33, further comprising an expandable insert configured to be insertable at least partially within the cannula lumen to drive expansion of the expandable region.

37. A method of treating a patient's spine, the method comprising:
positioning a distal end of a cannula proximate a target location along the patient's spine;
inserting a tool guide assembly at least partially within a cannula lumen of the cannula; and
positioning a spine surgical instrument proximate the target location, wherein positioning the spine surgical instrument proximate the target implant location includes inserting the spine surgical instrument through a guide feature of the tool guide assembly.

38. The method of claim 37 wherein inserting the tool guide assembly at least partially within the cannula lumen includes inserting the tool guide assembly until a tab of the tool guide assembly contacts the cannula.

39. The method of claim 37 wherein positioning the spine surgical instrument proximate the target location includes changing an orientation of the spine surgical instrument relative to the tool guide assembly.

40. The method of claim 37 wherein positioning the spine surgical instrument proximate the target implant location includes moving the spine surgical instrument relative to the tool guide assembly.

41. The method of claim 37 wherein inserting the tool guide assembly includes causing an expandable region of the cannula to expand.

42. The method of claim 37 wherein the spine surgical instrument includes an expandable intervertebral spacer, the method further comprising actuating the spine surgical instrument to cause the expandable intervertebral spacer to expand.

43. The method of claim 42 wherein actuating the spine surgical instrument includes actuating the spine surgical instrument while at least a portion of the expandable intervertebral spacer is positioned within the cannula.

44. The method of claim 37 wherein the spine surgical instrument is a first spine surgical instrument and tool guide assembly is a first tool guide assembly configured to correspond to the first spine surgical instrument, the method further comprising:
withdrawing the first tool guide assembly from the cannula lumen; and
positioning a second tool guide assembly at least partially within the cannula lumen, wherein the second tool guide assembly is configured to correspond to a second spine surgical instrument.

45. The method of claim 44 wherein positioning the second tool guide assembly includes positioning a tool guide assembly having a different configuration than the first tool guide assembly.

46. The method of claim 44 wherein positioning the second tool guide assembly includes positioning a distal end of the second tool guide assembly proximate the target location.

47. The method of claim 44 wherein withdrawing the first tool guide assembly includes withdrawing the first spine surgical instrument from the cannula lumen.

48. The method of claim 44 further comprising positioning the second spine surgical instrument at least partially within a second guide feature of the second tool guide assembly, wherein the second guide feature is configured to correspond to the second spine surgical instrument.

49. The method of claim 48 wherein positioning the second spine surgical instrument includes positioning the second spine surgical instrument proximate the target location.

50. The method of claim 48 wherein positioning the second spine surgical instrument includes positioning a spine surgical instrument different than the first spine surgical instrument.

51. The method of claim 37 wherein positioning the distal end of the cannula proximate the target location along the patient's spine includes inserting the cannula along a lateral lumbar interbody fusion path or a posterior lumbar interbody fusion path.

52. The method of claim 37 wherein positioning the distal end of the cannula proximate the target location along the patient's spine includes inserting the cannula along an anterior lumbar interbody fusion path, an oblique lumbar interbody fusion path, or a transforaminal lumbar interbody fusion path.

53. A spine surgical system, comprising:
an expandable intervertebral spacer;
a spine surgical instrument operably coupled to the expandable intervertebral spacer and actuable to transition the intervertebral spacer between an unexpanded configuration to an expanded configuration; and
a retractor including a retractor body, wherein the retractor body defines a retractor lumen configured to contain at least a portion of the expandable intervertebral spacer while the expandable intervertebral spacer transitions between the unexpanded configuration and the expanded configuration.

54. A method of treating a target location along a patient's spine, the method comprising:
positioning an intervertebral device within a retractor;
causing the intervertebral device to transition from an unexpanded configuration and an expanded configuration while the intervertebral device is positioned within the retractor;
causing the intervertebral device to transition from the expanded configuration toward the unexpanded configuration;
advancing the intervertebral device distally through an end of the retractor; and
causing the intervertebral device to transition from the unexpanded configuration to the expanded configuration to engage at least a portion of the patient's anatomy proximate the target location.

55. A retractor assembly, comprising:
a retractor body having a first end portion and a second end portion opposite the first end portion;
wherein the retractor body defines a retractor lumen extending through the retractor body between the first and second end portions and configured to receive one or more spine surgical instruments, and
wherein the retractor body includes an expandable region configured to expand in response to the insertion of at least one of the spine surgical instruments.
